(19) United States
(12) Patent Application Publication
(10) Pub. No.: US 2026/0232654 A1
(43) Pub. Date: Aug. 13, 2026

(54) TASTE-MASKING WITH A MULTI-COMPONENT PHYSICAL BARRIER

(71) Applicant: BAYER HEALTHCARE LLC, Whippany, NJ (US)

(72) Inventors: Reginald D. BRADLEY, Bedminster, NJ (US); Debanjan DAS, Morristown, NJ (US); Gerald MEISEL, Budd Lake, NJ (US); Tejas KEKATPURE, Edison, NJ (US)

(21) Appl. No.: 19/151,018

(22) PCT Filed: Jan. 26, 2024
(86) PCT No.: PCT/US2024/013210

§ 371 Date: Jul. 25, 2025

Related U.S. Application Data
(60) Provisional application No. 63/441,612, filed on Jan. 27, 2023.

(51) Int. Cl. A61K 31/485; A61K 9/00; A61K 47/10; A61K 47/12; A61K 47/18; A61K 47/26; A61K 47/36; A61K 47/40

(52) U.S. Cl. CPC A61K 31/485; A61K 9/0053; A61K 47/10; A61K 47/12; A61K 47/183; A61K 47/26; A61K 47/36; A61K 47/40

(57) ABSTRACT

Provided are liquid, solid, and semi-solid oral pharmaceutical compositions comprising a multi-component taste-masking physical barrier. The oral pharmaceutical compositions comprise an active pharmaceutical ingredient or a pharmaceutically acceptable salt thereof, one or more pharmaceutically acceptable excipients, a pH modifying agent, and the multi-component taste-masking physical barrier comprising one or more of an ion exchange resin, a cyclodextrin, and a hydrocolloid.

### CROSS-REFERENCE TO RELATED APPLICATIONS

This application claims priority to and benefit of U.S. Provisional Patent Application No. 63/441,612, filed Jan. 27, 2023, which is hereby incorporated by reference in its entirety.

### FIELD

The present disclosure generally relates to pharmaceutical compositions, and more particularly, to techniques for taste-masking pharmaceutical compositions.

### BACKGROUND

Many active pharmaceutical ingredients (APIs) are known to exhibit unpleasant organoleptic properties if allowed to dissolve in the oral cavity. For example, many APIs, and particularly those that are ionic, can impart sensations such as bitter taste, burning sensations, and numbing. Such unpleasant organoleptic feels or sensations can make it difficult for some patients to take a medication with the API(s) causing the unpleasant organoleptic feels or sensations. Difficulty taking such medication can make it difficult to overcome the health condition or ailment for which the medication is designed to alleviate.

### SUMMARY

Described herein are pharmaceutical compositions comprising a taste-masking system, and methods of making the same. Specifically, the pharmaceutical compositions provided herein include a system of excipients that are designed, when used together at specific amounts, to reduce the amount of sweetener required in the composition (which is sometimes used in higher quantities to balance out any unpleasant taste imparted by the active pharmaceutical ingredient (API). As described above, many orally-administered APIs provide an unpleasant organoleptic feel. This can make it difficult to ensure patient compliance. For example, ionic APIs can impart a bitter taste.

Accordingly, provided herein are pharmaceutical compositions comprising a particular system of excipient components that can reduce any unpleasant taste provided by the API for the time during which the composition is within the patient's mouth. Specifically, the taste-masking system forms a physical barrier between the API of the pharmaceutical composition and the gustatory calyculi (tastebuds) of a patient for a predetermined amount of time. The taste-masking system includes two or more of an ion exchange resin, a cyclodextrin, and a hydrocolloid. In some embodiments, the taste-masking system comprises one of each of an ion exchange resin, a cyclodextrin, and a hydrocolloid. The system of two or three components forms the physical barrier by entrapping the APIs and causing the pharmaceutical composition to slow contact between the API and the taste buds.

In some embodiments, provided is an oral pharmaceutical composition comprising: an active pharmaceutical ingredient or a pharmaceutically acceptable salt thereof; a pH modifying agent; at least two of an ion exchange resin, a cyclodextrin, and a hydrocolloid; and one or more pharmaceutically acceptable excipients.

In some embodiments of the oral pharmaceutical composition, the oral pharmaceutical composition comprises: an active pharmaceutical ingredient or a pharmaceutically acceptable salt thereof; a pH modifying agent; an ion exchange resin; a cyclodextrin; and a hydrocolloid.

In some embodiments of the oral pharmaceutical composition, the pH modifying agent comprises a buffering agent.

In some embodiments of the oral pharmaceutical composition, the pH modifying agent comprises one or more of lactic acid, acetic acid, citric acid, tartaric acid, phosphoric acid, or carbonic acid, or any pharmaceutically acceptable salt thereof.

In some embodiments of the oral pharmaceutical composition, if the pharmaceutical composition is a liquid dosage form, the pharmaceutical composition has a pH from about pH 4 to about pH 7; or if the pharmaceutical composition is a solid or semi-solid dosage form, the pharmaceutical composition has a pH from about pH 4 to about pH 7.

In some embodiments of the oral pharmaceutical composition, the ion exchange resin is a cationic exchange resin.

In some embodiments of the oral pharmaceutical composition, the ion exchange resin is an anionic exchange resin.

In some embodiments of the oral pharmaceutical composition, the cyclodextrin comprises one or more of alpha-cyclodextrin, beta-cyclodextrin, or gamma-cyclodextrin.

In some embodiments of the oral pharmaceutical composition, the cyclodextrin is beta-cyclodextrin.

In some embodiments of the oral pharmaceutical composition, the cyclodextrin is hydroxypropyl beta-cyclodextrin.

In some embodiments of the oral pharmaceutical composition, the hydrocolloid is pectin, carrageenan, xanthan gum, gellan gum, or any combination thereof.

In some embodiments of the oral pharmaceutical composition, the hydrocolloid is pectin, xanthan gum, guar gum, or a combination thereof.

In some embodiments of the oral pharmaceutical composition, the molar ratio of ion exchange resin to active pharmaceutical ingredient is at least about 0.5:1.

In some embodiments of the oral pharmaceutical composition, the (molar/weight) ratio of cyclodextrin to active pharmaceutical ingredient is at least about 4:1.

In some embodiments of the oral pharmaceutical composition, the (molar/weight) ratio of hydrocolloid to active pharmaceutical ingredient is at least about 0.5:1.0.

In some embodiments of the oral pharmaceutical composition, the active pharmaceutical ingredient or a pharmaceutically acceptable salt thereof is present in both an ionized form and in a non-ionized form, and wherein the amount of the ionized form of the active pharmaceutical ingredient or a pharmaceutically acceptable salt thereof present in the pharmaceutical composition is less than the amount of the non-ionized form.

In some embodiments of the oral pharmaceutical composition, the molar ratio of non-ionized active pharmaceutical ingredient to ionized active pharmaceutical ingredient is at least about 7,000 to 1 in synthetic saliva solution.

In some embodiments of the oral pharmaceutical composition, the pharmaceutical composition comprises less of the ionized form of the active pharmaceutical ingredient or a pharmaceutically acceptable salt thereof as compared to a reference pharmaceutical composition, and wherein the reference pharmaceutical composition does not contain at least two of a pH modifying agent, ion exchange resin, cyclodextrin and hydrocolloid.

In some embodiments of the oral pharmaceutical composition, the oral pharmaceutical composition further comprises one or more of a sweetener, a flavorant, a preservative, an antioxidant, a chelating agent, a viscosity modifying agent, a suspending agent, a pigment, or a colorant, or any combinations thereof.

In some embodiments of the oral pharmaceutical composition, the pharmaceutical composition has an immediate release profile.

In some embodiments of the oral pharmaceutical composition, the pharmaceutical composition has a delayed, sustained, or enteric release profile.

In some embodiments of the oral pharmaceutical composition, the pharmaceutical composition is a liquid dosage form.

In some embodiments of the oral pharmaceutical composition, the liquid dosage form is a solution, a syrup, or a suspension.

In some embodiments of the oral pharmaceutical composition, the pharmaceutical composition comprises at least about 0.1% wt. % ion exchange resin.

In some embodiments of the oral pharmaceutical composition, the pharmaceutical composition comprises at least about 0.2 wt. % cyclodextrin.

In some embodiments of the oral pharmaceutical composition, the pharmaceutical composition comprises at least about 0.1 wt. % hydrocolloid.

In some embodiments of the oral pharmaceutical composition, the pharmaceutical composition is a solid dosage form.

In some embodiments of the oral pharmaceutical composition, the solid dosage form is a tablet, a pellet, a bead, a powder, granules, or a lozenge.

In some embodiments of the oral pharmaceutical composition, the tablet is a swallow tablet a chewable tablet, a sublingual tablet, a buccal tablet, an orally disintegrating tablet, an orally disintegrating film strip, or an effervescent tablet.

In some embodiments of the oral pharmaceutical composition, the pharmaceutical composition comprises at least about 2 wt. % ion exchange resin.

In some embodiments of the oral pharmaceutical composition, the pharmaceutical composition comprises at least about 2 wt. % cyclodextrin.

In some embodiments of the oral pharmaceutical composition, the pharmaceutical composition comprises at least about 5 wt. % hydrocolloid.

In some embodiments of the oral pharmaceutical composition, the pharmaceutical composition is a semi-solid dosage form.

In some embodiments of the oral pharmaceutical composition, the semi-solid dosage form is a soft chew tablet or a chewable gel.

In some embodiments of the oral pharmaceutical composition, the pharmaceutical composition comprises at least about 2 wt. % ion exchange resin.

In some embodiments of the oral pharmaceutical composition, the pharmaceutical composition comprises at least about 2 wt. % cyclodextrin.

In some embodiments of the oral pharmaceutical composition, the pharmaceutical composition comprises at least about 5 wt. % hydrocolloid.

In some embodiments of the oral pharmaceutical composition, the active pharmaceutical ingredient or a pharmaceutically acceptable salt thereof comprises one or more of naproxen or a pharmaceutically acceptable salt thereof, diphenyhydramine or a pharmaceutically acceptable salt thereof, dextromethorphan or a pharmaceutically acceptable salt thereof, phenylephrine or a pharmaceutically acceptable salt thereof.

In some embodiments of the oral pharmaceutical composition, the active pharmaceutical ingredient or a pharmaceutically acceptable salt thereof is dextromethorphan or a pharmaceutically acceptable salt thereof.

In some embodiments of the oral pharmaceutical composition, the active pharmaceutical ingredient or a pharmaceutically acceptable salt thereof is a combination of diphenhydramine or a pharmaceutically acceptable salt thereof and phenylephrine or a or a pharmaceutically acceptable salt thereof.

In some embodiments, provided is a method of preparing an oral pharmaceutical composition according to any of the oral pharmaceutical composition embodiments described above, wherein the pharmaceutical composition is a liquid dosage form, comprising: combining an active pharmaceutical ingredient or a pharmaceutically acceptable salt thereof, an ion exchange resin, a cyclodextrin, and water to provide a mixture; homogenizing the mixture; adding a pH modifying agent to the mixture; and adding a hydrocolloid and one or more pharmaceutically acceptable excipients to the mixture, thereby providing the pharmaceutical composition in a liquid dosage form.

In some embodiments of the method, the one or more pharmaceutically acceptable excipients is a viscosity modifying agent, a suspending agent, or a combination thereof.

In some embodiments, provided is a method of preparing an oral pharmaceutical composition according to any of the oral pharmaceutical composition embodiments described above, wherein the pharmaceutical composition is a solid dosage form, comprising: combining an active pharmaceutical ingredient or a pharmaceutically acceptable salt thereof, a cyclodextrin, an ion exchange resin, and water to provide a first mixture; homogenizing the first mixture; adding a pH modifying agent to the first mixture; and adding a hydrocolloid and one or more pharmaceutically acceptable excipients to the first mixture to provide a second mixture; drying, and optionally milling, the second mixture, thereby providing the pharmaceutical composition in a solid dosage form, wherein the solid dosage form is a powder or granules.

In some embodiments of the method, the one or more pharmaceutically acceptable excipients is a viscosity modifying agent, a suspending agent, or a combination thereof.

In some embodiments of the method, the method further comprises adding one or more pharmaceutically acceptable excipients to the granules or powder in a tableting mixture, and compressing the pharmaceutical composition to provide the pharmaceutical composition in solid dosage form, wherein the solid dosage form is a tablet.

In some embodiments of the method, the tablet is a swallow tablet a chewable tablet, a sublingual tablet, a buccal tablet, an orally disintegrating tablet, or an effervescent tablet.

In some embodiments, provided is a method of preparing an oral pharmaceutical composition according to any of the oral pharmaceutical composition embodiments described above, wherein the pharmaceutical composition is a solid or semi-solid dosage form, comprising: combining an active pharmaceutical ingredient or a pharmaceutically acceptable salt thereof, an ion exchange resin, a cyclodextrin, and water to provide a first mixture; homogenizing the first mixture; adding a pH modifying agent to the first mixture; adding a hydrocolloid and one or more pharmaceutically acceptable excipients to the first mixture provide a second mixture; heating the second mixture; and molding the second mixture to form the pharmaceutical composition in a solid or semi-solid dosage form.

In some embodiments of the method, the one or more pharmaceutically acceptable excipients is a viscosity modifying agent, a suspending agent, or a combination thereof.

In some embodiments of the method, the solid or semi-solid dosage form is a lozenge, a soft chew tablet, an orally disintegrating film strip, or a chewable gel.

In some embodiments of the method, the cyclodextrin is combined with the active pharmaceutical ingredient or a pharmaceutically acceptable salt thereof and water prior to the resin.

In some embodiments of the method, the cyclodextrin and ion exchange resin are combined with the active pharmaceutical ingredient or a pharmaceutically acceptable salt thereof and water at the same time.

In some embodiments, provided is a method for treating cough and cold, comprising administering an oral pharmaceutical composition according to any of the oral pharmaceutical composition embodiments described above, to a human in need thereof, wherein the active pharmaceutical ingredient is dextromethorphan or a pharmaceutically acceptable salt thereof, phenylephrine or a pharmaceutically acceptable salt thereof, diphenhydramine or a pharmaceutically acceptable salt thereof, or a combination thereof.

In some embodiments, provided is a method for treatment of pain and/or fever, comprising administering an oral pharmaceutical composition according to any of the oral pharmaceutical composition embodiments described above, to a human in need thereof, wherein the active pharmaceutical ingredient is naproxen or a pharmaceutically acceptable salt thereof, phenylephrine or a pharmaceutically acceptable salt thereof, diphenhydramine or a pharmaceutically acceptable salt thereof, or a combination thereof.

In some embodiments, provided is a method for treatment of hay fever and/or upper respiratory allergies, comprising administering an oral pharmaceutical composition according to any of the oral pharmaceutical composition embodiments described above, to a human in need thereof, wherein the active pharmaceutical ingredient is diphenhydramine or a pharmaceutically acceptable salt thereof.

In some embodiments, any one or more of the features, characteristics, or elements discussed above with respect to any of the embodiments may be incorporated into any of the other embodiments mentioned above or described elsewhere herein.

### BRIEF DESCRIPTION OF THE FIGURES

FIG. 1 shows a depiction of a direct approach process by which a multi-component taste-masking system interacts with an API to form a taste-masked pharmaceutical composition, according to some embodiments;

FIG. 2 shows a depiction of an indirect approach process by which a multi-component taste-masking system interacts with an API to form a taste-masked pharmaceutical composition, according to some embodiments;

FIG. 3A shows a chemical structure of ion exchange resin, according to some embodiments;

FIG. 3B shows a chemical structure of cyclodextrin, according to some embodiments;

FIG. 3C shows chemical structures of hydrocolloid, according to some embodiments;

FIG. 3D shows chemical structures of hydrocolloid, according to some embodiments; and

FIG. 4 shows an example of a process for preparing a liquid pharmaceutical formulation comprising a multi-component taste-masking physical barrier, according to some embodiments.

### DETAILED DESCRIPTION

Described herein are pharmaceutical compositions comprising a taste-masking system for minimizing or eliminating the unpleasant taste often caused by active pharmaceutical ingredients (APIs), and methods of making the same. APIs that impart unpleasant organoleptic properties may increase patient noncompliance. For example, ionic APIs in particular can cause a bitter taste for patients, making it difficult for them to take an oral dosage form comprising ionic APIs.

Accordingly, the pharmaceutical compositions provided herein include a multi-component taste-masking system. The multi-component taste-masking system includes a pH modifier and two or more of an ion exchange resin, a cyclodextrin, and a hydrocolloid. In some embodiments, the taste-masking system includes a pH modifier and all three of an ion exchange resin, a cyclodextrin, and a hydrocolloid.

The multi-component taste-masking system forms a physical barrier between the API of the pharmaceutical composition and the taste buds of a patient. An effective physical taste-masking system formed by the multi-component taste-masking system described herein will decrease the ion channel function and/or reduce the transduction process, which are the functions that cause chemical signals to transcribe electrical charges within the taste buds by relaying depolarization and the release of neurotransmitters that elicit taste.

The process by which the multi-component taste-masking system may be applied to an API can include granulation, film coating, spray drying, and/or direct mixing. The pharmaceutical compositions described herein can include liquids, solids, semi-solids, and derivatives thereof such as, but not limited to, gummies, strips, and chewing gum. In some embodiments, the pharmaceutical composition is a liquid. A liquid dosage form can include a solution, a syrup, or a suspension. In some embodiments, the dosage form is a solid. For example, a solid dosage form may comprise a tablet, a pellet, a bead, a powder, granules, or a lozenge. In some embodiments, the dosage form of the pharmaceutical composition is a tablet. For example, the tablet can comprise a swallowable tablet, a chewable tablet, a sublingual tablet, a buccal tablet, an orally disintegrating tablet, an orally disintegrating film strip, or an effervescent tablet. In some embodiments, the dosage form of the pharmaceutical composition is a semi-solid dosage form. For example, a semi-sold dosage form may comprise a soft chew tablet or a chewable gel. In some embodiments, the multi-component physical barrier taste-masking system provided herein may be configured to mask the taste of multiple APIs within a single pharmaceutical composition.

### Pharmaceutical Compositions Comprising a Multi-Component Taste-Masking Physical Barrier System

Described below are exemplary pharmaceutical compositions comprising a multi-component taste-masking physical barrier, according to some embodiments provided herein. The pharmaceutical compositions comprise one or more active pharmaceutical ingredients (APIs), a pH modifier, and two or more of an ion exchange resin, a cyclodextrin, and a hydrocolloid. In some embodiments, the pharmaceutical composition comprises one or more APIs, a pH modifier, an ion exchange resin, a cyclodextrin, and a hydrocolloid. The pharmaceutical composition can further include one or more pharmaceutically-acceptable excipients.

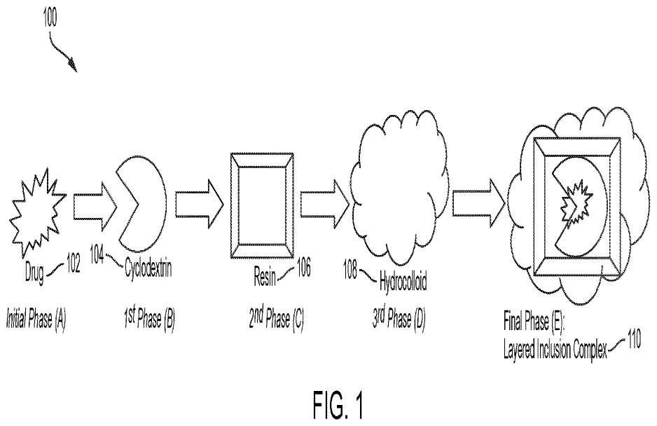

FIG. 1 shows a depiction of a direct approach process 100 by which a multi-component taste-masking system interacts with an API to form a taste-masked pharmaceutical composition. As shown, FIG. 1 includes an API 102, resin 104, cyclodextrin 106, hydrocolloid 108, and a taste-masked API 110. Also shown is a buffering agent 112.

This direct approach shown in FIG. 1 demonstrates that most APIs have the ability to be taste-masked using the multi-component physical barrier system described herein, depending on the API's chemical moiety, ionic charges, and/or solubility. The size of the API structure will rely on its ability to be inclusion or complexation process and/or a combination of them both in order to provide reduction of their bitter attribute.

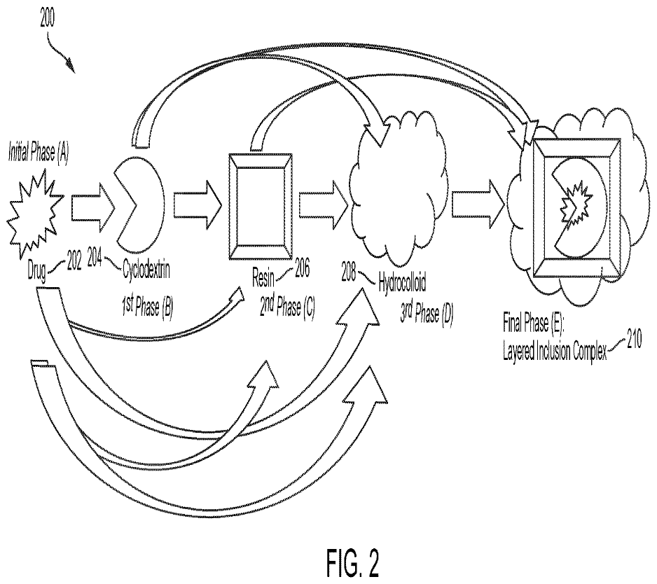

FIG. 2 shows a depiction of an indirect approach process 200 by which a multi-component taste-masking system interacts with an API to form a taste-masked pharmaceutical composition. As shown, FIG. 2 includes an API 202, cyclodextrin 206, resin 204, hydrocolloid 208, and a taste-masked API 210. Also shown is a buffering agent 212.

This indirect approach shown in FIG. 2 demonstrates that APIs can be used in an inclusion process by allowing ion exchange resin to deliver drugs based on their principles of charge. Glucose monomers and polymer (as in beta cyclodextrin) can entrap the resin substrate, thus reducing the bitter attribute from being exposed. Then, cyclodextrin can be used as a cup to surround the glucose monomers. Finally, adding hydrocolloid can provide a further layer of protection. In some embodiments, by forming a gel in the presence of water, these four steps work synergically with one another for the success of this technology.

Hydrogen (pH) resin, the cyclodextrin, the hydrocolloid, one or more bitter tasting APIs, and commonly used excipients will provide a protective coating around the drug dosage form in order to taste mask the taste, its grittiness, and mouth feel, due to the potential of the hydrogen (pH) resin. The combination of these components effectively taste-mask the bitter-tasting API(s) and can provide rapid relief for a user.

As indicated by the arrows in FIG. 2, the process can add cyclodextrin directly to the API, and then the resin and hydrocolloid, in that order. In some embodiments, the process can add resin to the API, and then the hydrocolloid, in that order. In some embodiments, the process can add hydrocolloid to the API, and then cyclodextrin and resin, in that order.

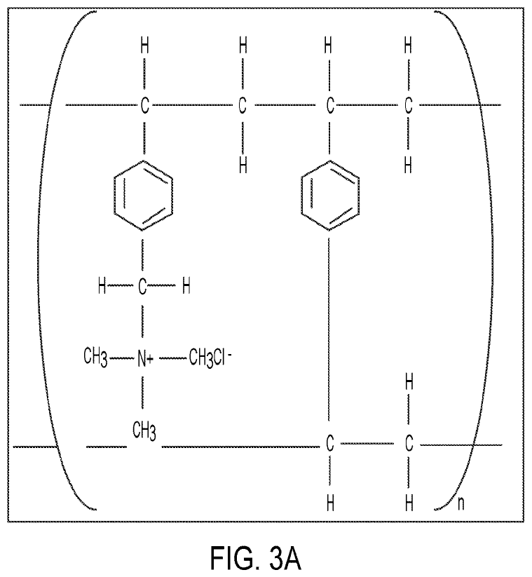

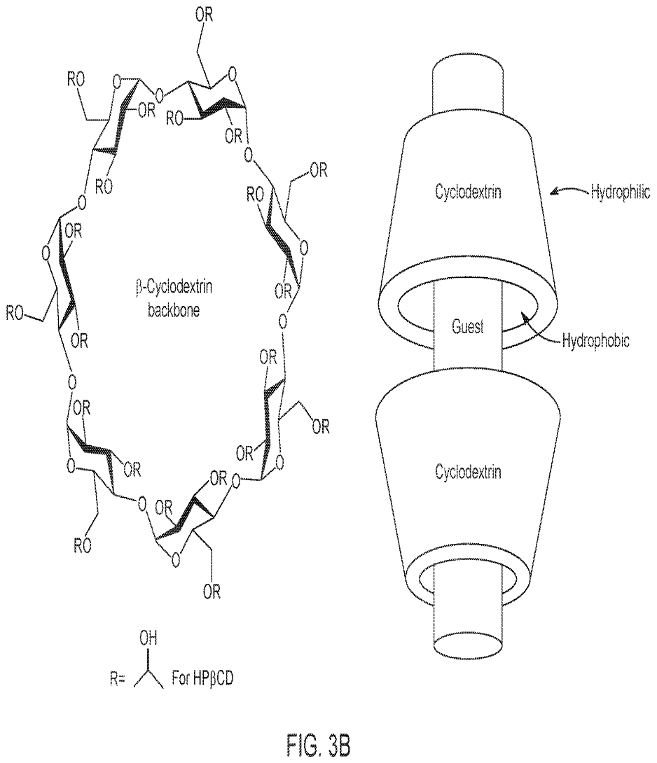

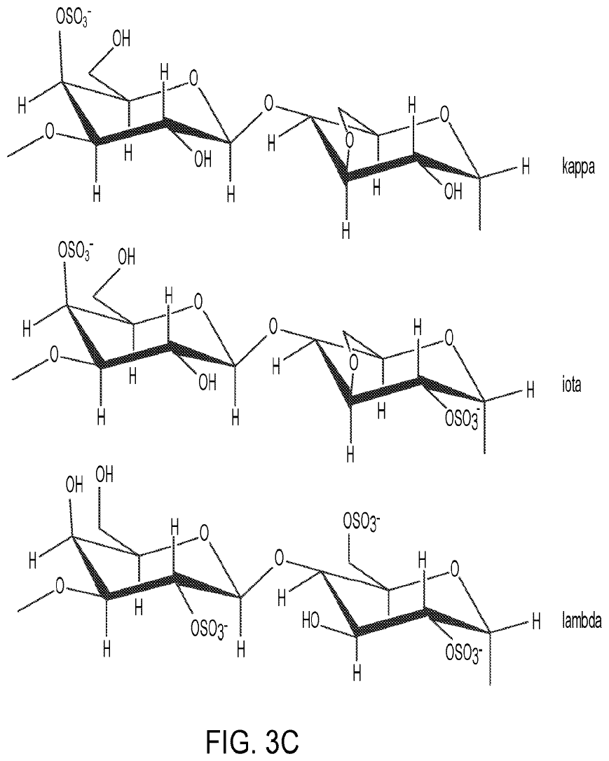

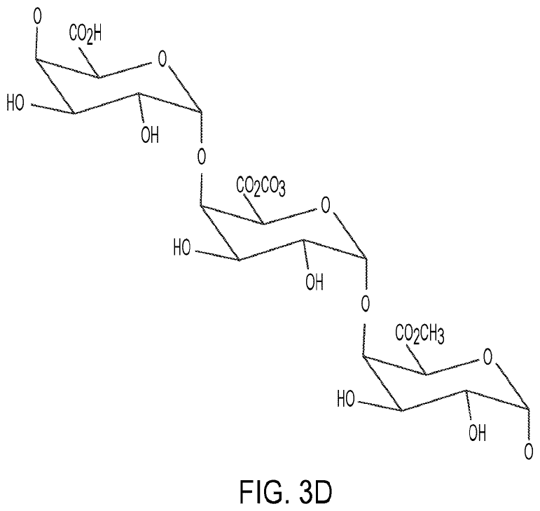

FIG. 3A shows a chemical structure of ion exchange resin, according to some embodiments, FIG. 3B shows a chemical structure of cyclodextrin, according to some embodiments, and FIGS. 3C and 3D show chemical structures of the hydrocolloid, according to some embodiments.

Although FIG. 1 and FIG. 2 show the components in the following order: API-resin-cyclodextrin-hydrocolloid and API-cyclodextrin, resin-hydrocolloid, respectively, the process is not limited to adding the components in either order, nor is the pharmaceutical composition limited to the layers of the multi-component taste-masking physical barrier being added to the one or more APIs in this order. For example, in some embodiments, two or more of the components may be added at the same time. The cyclodextrin may be added prior to the resin and hydrocolloid. The resin may be added prior to the cyclodextrin and hydrocolloid. The hydrocolloid may be added prior to the resin and cyclodextrin.

In some embodiments, the API is first incorporated into the cyclodextrin. The cyclodextrin-coated API is then incorporated into the resin. Finally, in some embodiments, the API-cyclodextrin-resin matrix is sealed with the hydrocolloid. A pH modifier can be used to adjust the pH of the composition for stabilization.

In some embodiments, a pharmaceutical composition comprising the multi-component taste-masking physical barrier provided herein may have two or more discrete single-component layers. For example, a resin may be layered directly over the API, followed by a layer of the cyclodextrin and a layer of the hydrocolloid. In some embodiments, a resin may be layered directly over the API, followed by a layer of the hydrocolloid and a layer of the cyclodextrin. In some embodiments, a cyclodextrin may be layered directly over the API, followed by a layer of the resin and a layer of the hydrocolloid. In some embodiments, a cyclodextrin may be layered directly over the API, followed by a layer of the hydrocolloid and a layer of the resin. In some embodiments, a hydrocolloid may be layered directly over the API, followed by a layer of the cyclodextrin and a layer of the resin. In some embodiments, a hydrocolloid may be layered directly over the API, followed by a layer of the resin and a layer of the cyclodextrin. The taste-masking components may be added in any order, so long as the pH of the composition is adjusted appropriately in the final step.

In some embodiments, the physical barrier may comprise one or more layers comprising a blend of two or more components. For example, a first layer may comprise the cyclodextrin and the resin. A second layer may comprise the resin only. In some embodiments, the first layer may comprise cyclodextrin only, and a second layer may comprise a mixture of hydrocolloid and resin. In some embodiments, the physical barrier may comprise a single layer comprising a blend of the resin, cyclodextrin, and/or hydrocolloid. Specifically, the physical barrier is a multiple encapsulation/inclusion where various ingredients are incorporated into each other. The overall system pH is maintained such that the physical barrier maintains at least 5 times the non-ionized form of the API as compared to the ionized form of the API. Optimizing the pH helps us to encapsulate the bitter tasting API better. Final pH is always adjusted in the end to favor the predominance of the non-ionized form of the API over the ionized form of the API. The ratio of ionized to non-ionized API can determine the level of bitterness perception of the formula. Thus, pH control can also improve the taste of the composition.

In some embodiments, the taste-masked pharmaceutical compositions described herein may demonstrate a slight change in reaction time as compared to that of untreated APIs. However, the speed at which a patient who ingests a taste-masked pharmaceutical composition according to embodiments here can receive relief from various conditions such as headaches, muscle aches, tendonitis, dental pain, menstrual cramps, pediatric ailments, etc., (and depending on the specific API of the pharmaceutical composition) will stay consistent as compared to that of a non-taste-masked pharmaceutical composition.

Active Pharmaceutical Ingredients: The pharmaceutical compositions provided herein include one or more APIs. In some embodiments, the one or more APIs of the pharmaceutical composition can provide an unpleasant organoleptic feel to a patient. For example, some APIs, and in particular ionic APIs, can taste bitter to a user. However, the multi-component taste-masking physical barrier can also be used with APIs that do not provide an unpleasant organoleptic feel.

In some embodiments, a liquid dosage form pharmaceutical composition may comprise 0.05-1 wt. % one or more APIs. In some embodiments, a liquid dosage form pharmaceutical composition may comprise less than or equal to 1, 0.9, 0.8, 0.7, 0.6, 0.5, 0.4, 0.3, 0.2, or 0.1 wt. % one or more APIs. In some embodiments, a liquid dosage form pharmaceutical composition may comprise greater than or equal to 0.05, 0.1, 0.2, 0.3, 0.4, 0.5, 0.6, 0.7, 0.8, or 0.9 wt. % one or more APIs.

In some embodiments, a solid dosage form pharmaceutical composition may comprise 50-80 wt. % one or more APIs. In some embodiments, a solid dosage form pharmaceutical composition may comprise less than or equal to 80, 75, 70, 65, 60, or 55 wt. % one or more APIs. In some embodiments, a solid dosage form pharmaceutical composition may comprise greater than or equal to 50, 55, 60, 65, 70, or 75 wt. % one or more APIs.

The one or more APIs may be, in some embodiments, a pharmaceutically acceptable salt of a particular API. The one or more APIs may also be present in an ionized form and/or an non-ionized form. In some embodiments, when the API is present in both an ionized form and a non-ionized form, the ionized form is present in quantity less than that of the non-ionized form. In some embodiments, the amount of the ionized form of API present in saliva (e.g., synthetic saliva solution) is less than that of a pharmaceutical composition without the multi-component taste-masking physical barrier described herein. In some embodiments, the one or more APIs are water-soluble.

In some embodiments, the molar ratio of the non-ionized form of Dextromethorphan HBr to the ionized form of Dextromethorphan HBr is approximately 700,000:1 (specifically 707,945.78:1) in synthetic saliva solution according to the Henderson-Hasselbach equation at a pH of 4. In some embodiments, the molar ratio of the non-ionized form of Phenylephrine HCl to the ionized form of Phenylephrine HCl is approximately 90,000:1 (specifically 93,325.43:1) in synthetic saliva solution according to the Henderson-Hasselbach equation at a pH of 4. In some embodiments, the molar ratio of the non-ionized form of Diphenhydramine HCl to the ionized form of Diphenhydramine HCl is approximately 7,000:1 (specifically 7,413.10:1) in synthetic saliva solution according to the Henderson-Hasselbach equation at a pH of 5. In some embodiments, the molar ratio of the non-ionized form of Doxylamine succinate to the ionized form of Doxylamine succinate is approximately 7,000:1 (specifically 7,413.10:1) in synthetic saliva solution according to the Henderson-Hasselbach equation at a pH of 5.

Suitable APIs can include, but are not limited to, non-steroidal anti-inflammatory drugs (e.g., naproxen), antihistamines (e.g., diphenyhydramine, doxylamine), antitussives (e.g., dextromethorphan), decongestants (e.g., phenylephrine), expectorants (e.g. guaifenesin), or pharmaceutically acceptable salts thereof.

pH Modifying Agent: In some embodiments, the pharmaceutical composition described herein may be prepared by first modifying the pH of the one or more APIs with a pH modifying agent, and then applying the multi-component taste-masking physical barrier to form the physical barrier around the one or more APIs. In some embodiments, the pH modifying agent may be a buffering agent. In some embodiments, the pH modifying agent may comprise one or more of lactic acid, acetic acid, citric acid, tartaric acid, phosphoric acid, or carbonic acid. A buffering, or pH modifying, system may be used to maintain stability in the formulation.

In some embodiments, a liquid dosage form pharmaceutical composition may comprise 0.05-1 wt. % pH modifying agent. In some embodiments, a liquid dosage form pharmaceutical composition may comprise less than or equal to 1, 0.9, 0.8, 0.7, 0.6, 0.5, 0.4, 0.3, 0.2, or 0.1 wt. % pH modifying agent. In some embodiments, a liquid dosage form pharmaceutical composition may comprise greater than or equal to 0.05, 0.1, 0.2, 0.3, 0.4, 0.5, 0.6, 0.7, 0.8, or 0.9 wt. % % pH modifying agent.

In some embodiments, a solid/semi-solid dosage form pharmaceutical composition may comprise 0.01-0.5 wt. % pH modifying agent. In some embodiments, a solid/semi-solid dosage form pharmaceutical composition may comprise less than or equal to 0.5, 0.4, 0.3, 0.2, or 0.1% pH modifying agent. In some embodiments, a solid/semi-solid dosage form pharmaceutical composition may comprise greater than or equal to 0.05, 0.1, 0.2, 0.3, or 0.4% pH modifying agent.

In some embodiments, the pH of the pharmaceutical composition may be from 3-9. In some embodiments, the pH of the pharmaceutical composition may be less than or equal to 9, 8, 7, 6, 5, or 4. In some embodiments, the pH of the pharmaceutical composition may be greater than or equal to 3, 4, 5, 6, 7, or 8. In some embodiments, the dosage form of a pharmaceutical composition having a pH of 3-9 is a liquid dosage form. In some embodiments, the dosage form of a pharmaceutical composition having a pH of 3-9 is a solid or semi-solid dosage form, and the pH is measured by inserting a probe directly into the product.

Cyclodextrin: In some embodiments, a pharmaceutical composition described herein may include a cyclodextrin. The cyclodextrin may be used specifically as a component of the multi-component taste-masking physical barrier of the pharmaceutical composition. In some embodiments, the cyclodextrin comprises one or more of alpha-cyclodextrin, beta-cyclodextrin, or gamma-cyclodextrin. In some embodiments, the cyclodextrin comprises a beta-cyclodextrin comprising hydroxypropyl beta-cyclodextrin. A suitable commercially-available hydrocolloid includes Kleptose®.

In some embodiments, the molar/weight ratio of cyclodextrin to API in a pharmaceutical composition may be 3:1 to 5:1. In some embodiments, the molar/weight ratio of cyclodextrin to API may be less than or equal to 5:1, 4.5:1, 4:1, or 3.5:1. In some embodiments, the molar/weight ratio of cyclodextrin to API may be greater than or equal to 3:1, 3.5:1, 4:1, or 4.5:1. The exact molar/weight ratio of cyclodextrin to API in a pharmaceutical composition according to embodiments described herein may be dependent upon the dosage form (i.e., solid, semisolid, or liquid) and/or the specific APIs used. For example, the ratio of cyclodextrin to API is generally highest for liquid formulations, followed by semi-solid formulations, and then solid formulations.

In some embodiments, a liquid dosage form pharmaceutical composition may have from 0.01-1 wt. % cyclodextrin. In some embodiments, a liquid dosage form pharmaceutical composition may have less than or equal to 1, 0.9. 0.8, 0.7, 0.6, 0.5, 0.4, 0.3, 0.2, 0.1, or 0.05 wt. % cyclodextrin. In some embodiments, a liquid dosage form pharmaceutical composition may have more than or equal to 0.01, 0.05, 0.1, 0.2, 0.3, 0.4, 0.5, 0.6, 0.7, 0.8, or 0.9 wt. % cyclodextrin.

In some embodiments, a solid/semi-solid dosage form pharmaceutical composition may comprise 0.01-10 wt. % cyclodextrin. In some embodiments, a solid/semi-solid dosage form pharmaceutical composition may comprise less than or equal to 10, 9, 8, 7, 6, 5, 4, 3, 2, 1, or 0.5 wt. % cyclodextrin. In some embodiments, a solid/semi-solid dosage form pharmaceutical composition may comprise greater than or equal to 0.01, 1, 2, 3, 4, 5, 6, 7, 8, or 9 wt. % cyclodextrin.

Hydrocolloid: In some embodiments, a pharmaceutical composition described herein may include a hydrocolloid. The hydrocolloid may be used specifically as a component of the multi-component taste-masking physical barrier of the pharmaceutical composition. In some embodiments, the hydrocolloid may comprise one or more of pectin, carrageenan, xanthan gum, guar gum, or gellan gum.

In some embodiments, the molar/weight ratio of hydrocolloid to API in a pharmaceutical composition may be 0.1:1 to 2:1. In some embodiments, the molar/weight ratio of hydrocolloid to API may be less than or equal to 2:1, 1.5:1, 1:1, or 0.5:1. In some embodiments, the molar/weight ratio of hydrocolloid to API may be greater than or equal to 0.1:1, 0.5:1, 1:1, or 1.5:1. The exact molar/weight ratio of hydrocolloid to API in a pharmaceutical composition according to embodiments described herein may be dependent upon the dosage form (i.e., solid, semisolid, or liquid) and/or the specific APIs used. The ratio of hydrocolloid to API is highest for a liquid formulation, followed by a semi-solid formulation, and lowest for a solid formulation.

In some embodiments, a liquid dosage form or a solid/semi-solid dosage form pharmaceutical composition may comprise 0.01-20 wt. % hydrocolloid. In some embodiments, a pharmaceutical composition may comprise less than or equal to 20, 15, 10, 8, 6, 4, 2, 1, 0.9. 0.8, 0.7, 0.6, 0.5, 0.4, 0.3, 0.2, 0.1, or 0.05 wt. % hydrocolloid. In some embodiments, a pharmaceutical composition may comprise more than or equal to 0.01, 0.05, 0.1, 0.2, 0.3, 0.4, 0.5, 0.6, 0.7, 0.8, 0.9, 1, 2, 4, 6, 8, 10, or 15 wt. % hydrocolloid.

Ion Exchange Resin: In some embodiments, the pharmaceutical composition described herein includes an ion exchange resin. The ion exchange resin is one of the components used to form the multi-component taste-masking physical barrier. In some embodiments, the ion exchange resin may be a cationic exchange resin. For example, a suitable cationic exchange resin may include AmberLite IRP88. In some embodiments, the ion exchange resin may be an anionic exchange resin.

In some embodiments, a liquid dosage form pharmaceutical composition may comprise 0.01-10 wt. % ion exchange resin. In some embodiments, a liquid dosage form pharmaceutical composition may comprise less than or equal to 10, 9, 8, 7, 6, 5, 4, 3, 2, or 1 wt. % ion exchange resin. In some embodiments, a liquid dosage form pharmaceutical composition may comprise greater than or equal to 0.01, 1, 2, 3, 4, 5, 6, 7, 8, or 9 wt. % ion exchange resin.

In some embodiments, a solid/semi-solid dosage form pharmaceutical composition may comprise 1-15 wt. % ion exchange resin. In some embodiments, a solid/semi-solid dosage form pharmaceutical composition may comprise less than or equal to 15, 14, 13, 12, 11, 10, 9, 8, 7, 6, 5, 4, 3, or 2 wt. % ion exchange resin. In some embodiments, a solid/semi-solid dosage form pharmaceutical composition may comprise greater than or equal to 1, 2, 3, 4, 5, 6, 7, 8, 9, 10, 11, 12, 13, or 14 wt. % ion exchange resin.

In some embodiments, the molar ratio of ion exchange resin to API is 0.5:1 to 1.5:1. In some embodiments, the molar ratio of ion exchange resin to API is less than or equal to 1.5:1, 1.25:1, 1:1, or 0.75:1. In some embodiments, the molar ratio of ion exchange resin to API is greater than or equal to 0.5:1, 0.75:1, 1:1, or 1.25:1. The exact molar ratio of ion exchange resin to API in a pharmaceutical composition according to embodiments described herein may be dependent upon the dosage form (i.e., solid, semisolid, or liquid) and/or the specific APIs used. The ratio of ion exchange resin to API is highest for a liquid formulation, followed by a semi-solid formulation, and lowest for a solid formulation.

In some embodiments, a pharmaceutical composition may have from 0.01-1 wt. % ion exchange resin. In some embodiments, a pharmaceutical composition may have less than or equal to 1, 0.9. 0.8, 0.7, 0.6, 0.5, 0.4, 0.3, 0.2, 0.1, or 0.05 wt. % ion exchange resin. In some embodiments, a pharmaceutical composition may have more than or equal to 0.01, 0.05, 0.1, 0.2, 0.3, 0.4, 0.5, 0.6, 0.7, 0.8, or 0.9 wt. % ion exchange resin.

Further Excipients: In some embodiments, the pharmaceutical compositions provided herein can comprise excipients such as, but not limited to, a sweetener, a flavorant, a preservative, an antioxidant, a chelating agents, a viscosity modifying agent, a suspending agent, a pigment, or a colorant. The pharmaceutical compositions described herein can include any combinations of the above enumerated excipients.

Release Profile: In some embodiments, the pharmaceutical composition comprises an immediate release profile. In some embodiments, the pharmaceutical composition comprises a delayed release profile. In some embodiments, the pharmaceutical composition comprises a sustained release profile. In some embodiments, the pharmaceutical composition comprises an enteric release profile.

### Time of Taste-Masking

The multi-component physical taste-masking barrier described herein can be designed to taste-mask the API of the pharmaceutical composition for a predetermined amount of time. For example, the predetermined amount of time may be based on how long the pharmaceutical composition is expected to stay in the oral cavity (e.g., based on the dosage form). In some embodiments, the predetermined amount of time may be 1 second to 5 minutes. In some embodiments, the predetermined amount of time may be less than or equal to 5 minutes, 4 minutes, 3, minutes, 2 minutes, 1 minute, 45 seconds, 30 seconds, 20 seconds, 15 seconds, 10 seconds, or 5 seconds. In some embodiments, the predetermined amount of time may be greater than or equal to 1 second, 5, seconds, 10 seconds, 15 seconds, 20 seconds, 30 seconds, 45 seconds, 1 minute, 2 minutes, 3 minutes, or 4 minutes.

### Examples of Pharmaceutical Compositions Comprising a Multi-Component Taste-Masking Physical Barrier System

Below, Table 1 shows that four separate APIs, when mixed with purified water, do not provide taste-masking properties. These combinations are used as a control.

TABLE 1

| Factors | Category | Combinations |
|---|---|---|
| A | Ph Below 6.0 | AC, AD, AE. AF |
| B | Ph Above 7.0 | BC, BD, BE, BF |
| C | Diphenhydramine HCl | |
| D | Doxylamine succinate | |
| E | Dextromethorphan HBr | |
| F | Phenylephrine HCl | |

AC, AD, AE, AF, BC, BD, BE, BF don't successfully taste mask

Below, Table 2 shows that four separate APIs, when mixed with purified water and one of components C, D, or E, do not provide taste-masking properties. Thus, this table shows that a single-component taste-masking system comprising only one of an ion exchange resin, a cyclodextrin, or a hydrocolloid does not effectively mask the taste of an API.

TABLE 2

| Factors | Category | Combinations |
|---|---|---|
| A | Ph Below 6.0 | ACF, ACG, ACH, ACI, ADF, ADG, |
| B | Ph Above 7.0 | ADH. ADI, AEF, AEG, AEH, AEI, |
| C | Resin | (n = 12) |
| D | Cyclodextrin | BCF, BCG, BCH, BCI, BDF, BDG, |
| E | Hydrocolloid | BDH, BDI, BEF, BEG, BEH, BEI, |
| F | Diphenhydramine HCl | (n = 12) |
| G | Doxylamine succinate | |
| H | Dextromethorphan HBr | |
| I | Phenylephrine HCl | |

ACF, ACG, ACH, ACI, ADF, ADG, ADH. ADI, AEF, AEG, AEH, AEI, slightly tasted masked

Below, Table 3 shows that four separate APIs, when mixed with purified water and two of components C, D, or E, only provide slight taste-masking properties. Thus, this table shows that a multi-component taste-masking system comprising only two of an ion exchange resin, a cyclodextrin, or a hydrocolloid only slightly masks the taste of an API.

TABLE 3

| Factors | Category | Combinations |
|---|---|---|
| A | Ph Below 6.0 | ACDF, ACDG, ACDH, |
| B | Ph Above 7.0 | ACDI, (n = 4) |
| C | Resin | BCDF, BCDG, BCDH, |
| D | Cyclodextrin | BCDI (n = 4) |
| E | Hydrocolloid | |
| F | Guaifenesin | |
| G | Dextromethorphan HBr | |
| H | Diphenhydramine HCl | |
| I | Phenylephrine HCl | |

ACDF, ACDG, ACDH, ACDI, BCDF, BCDG, BCDH, BCDI slightly tasted masked

Below, Table 4 shows that four separate APIs, when mixed with purified water and three of components C, D, or E, provides adequate taste-masking properties. Thus, this table shows that a multi-component taste-masking system comprising all three of an ion exchange resin, a cyclodextrin, and a hydrocolloid adequately masks the taste of an API.

TABLE 4

| Factors | Category | Combinations |
|---|---|---|
| A | Ph Below 6.0 | ACDEF, ACDEG, ACDEH, |
| B | Ph Above 7.0 | ACDEI (n = 4) |
| C | Resin | BCDEF, BCDEG, BCDEH, |
| D | Cyclodextrin | BCDEI (n = 4) |
| E | Hydrocolloid | |
| F | Naproxen sodium | |
| G | Diphenhydramine HCl | |
| H | Phenylephrine | |
| I | Dextromethorphan HBr | |

ACDEF, ACDEG, ACDEH, ACDEI, BCDEF, BCDEG, BCDEH, BCDEI successfully tasted masked

Tables 5-12, below, show examples of pharmaceutical compositions according to some embodiments. As shown, the compositions of Tables 5-8 comprise all three of resin, cyclodextrin, and hydrocolloid. Specifically, Tables 5-7 show compositions for daytime formulations, and Table 8 shows a composition for a nighttime formulation. Tables 9 and 10 show pharmaceutical formulations that do not provide adequate taste-masking. Specifically, Table 9 shows a composition for a daytime formulation with a neutral pH (6-7) and Table 10 shows a composition for a nighttime formulation. The formulation of Table 11 is a daytime formula that does not comprise resin, but does have adequate taste-masking. Unlike that of Table 9, the pH of the formulation of Table 11 is adjusted to a pH of approximately 4.0 (using a buffer system of citric acid and sodium dihydrate) to compensate for the lack of resin as a taste-masking agent. Similarly, the formulation of Table 12, a nighttime formula, is also missing resin, but like that of the formulation of Table 11, the pH of the formulation of Table 12 is adjusted to a pH of 5.0 (using a buffer system of citric acid and sodium dihydrate) to compensate for the lack of resin as a taste-masking agent.

TABLE 5
Day Time Pharmaceutical Composition

| Ingredients | mg/dose | % |
|---|---|---|
| Dextromethorphan HBr | 20.00 | 0.13 |
| Resin | 40.00 | 0.26 |
| Cyclodextrin | 50.00 | 0.33 |
| Glycerin | 20.00 | 0.13 |
| Hydrocolloid | 20.00 | 0.13 |
| Citric Acid | 30.00 | 0.20 |
| Trisodium Citrate | 5.00 | 0.03 |
| Xanthan Gum | 30.00 | 0.20 |
| Sucralose | 25.00 | 0.17 |
| Flavor | 250.00 | 1.67 |
| Edetate Disodium | 2.50 | 0.02 |
| Sodium Benzoate | 30.00 | 0.20 |
| Color | 4.00 | 0.03 |
| Purified water | 14473.50 | 96.49 |
| 20 mg/20 mg 15000 mL dose Total | 15000.00 | 100 |

TABLE 6
Day Time Pharmaceutical Composition

| Ingredients | mg/dose | % |
|---|---|---|
| Guaifenesin | 200.00 | 1.30 |
| Resin | 40.00 | 0.26 |
| Cyclodextrin | 50.00 | 0.33 |
| Glycerin | 20.00 | 0.13 |
| Hydrocolloid | 20.00 | 0.13 |
| Citric Acid | 30.00 | 0.20 |
| Trisodium Citrate | 5.00 | 0.03 |
| Xanthan Gum | 30.00 | 0.20 |
| Sucralose | 25.00 | 0.17 |
| Flavor | 250.00 | 1.67 |
| Edetate Disodium | 2.50 | 0.02 |
| Sodium Benzoate | 30.00 | 0.20 |
| Color | 4.00 | 0.03 |
| Purified water | 14473.50 | 96.49 |
| 20 mg/20 mg 15000 mL dose Total | 15000.00 | 100 |

TABLE 7
Day Time Pharmaceutical Formulation

| Ingredients | Mg/dose | % |
|---|---|---|
| Naproxen Sodium | 220.00 | 68.69 |
| Cyclodextrin | 9.48 | 2.96 |
| Resin | 9.48 | 2.96 |
| Mannitol | 37.90 | 11.83 |
| Gelatin | 40.00 | 12.49 |
| Hydrocolloid | 20.00 | 6.24 |
| Citric Acid | 0.40 | 0.12 |
| Flavor | 0.30 | 0.09 |
| Sucralose | 1.60 | 0.50 |
| Pigment | 0.10 | 0.03 |
| Purified water | * | — |
| Total | 320.30 | 100 |

*water will evaporate off

TABLE 8
Night Time Pharmaceutical Composition

| Ingredients | mg/dose | % |
|---|---|---|
| Diphenhydramine HCl | 25.00 | 0.17 |
| Phenylephrine | 10.00 | 0.07 |
| Resin | 100.00 | 0.67 |
| Cyclodextrin | 45.00 | 0.30 |
| Glycerin | 20.00 | 0.13 |
| Hydrocolloid | 20.00 | 0.13 |
| Citric Acid | 30.00 | 0.20 |
| Xanthan Gum | 30.00 | 0.20 |
| Sucralose | 70.00 | 0.47 |
| Flavor | 600.00 | 4.00 |
| Edetate Disodium | 2.50 | 0.02 |
| Sodium Benzoate | 30.00 | 0.20 |
| Color | 10.00 | 0.07 |
| Purified water | 14007.50 | 93.38 |
| 25 mg/10 mg/20 mg15000/dose Total | 15000.00 | 100 |

TABLE 9
Day Time Pharmaceutical Composition

| Ingredients | mg/dose | % |
|---|---|---|
| Dextromethorphan HBr | 20.00 | 0.13 |
| Resin | XX | XX |
| Cyclodextrin | 50.00 | 0.33 |
| Glycerin | 20.00 | 0.13 |
| Hydrocolloid | 20.00 | 0.13 |
| Citric Acid | 30.00 | 0.20 |
| Trisodium Citrate | 5.00 | 0.03 |
| Xanthan Gum | 30.00 | 0.20 |
| Sucralose | 25.00 | 0.17 |
| Flavor | 250.00 | 1.67 |
| Edetate Disodium | 2.50 | 0.02 |
| Sodium Benzoate | 30.00 | 0.20 |
| Color | 4.00 | 0.03 |
| Purified water | 14512.50 | 96.75 |
| 20 mg/20 mg 15000 mL dose Total | 15000.00 | 100 |

TABLE 10
Night Time Pharmaceutical Composition

| Ingredients | mg/dose | % |
|---|---|---|
| Diphenhydramine HCl | 25.00 | 0.17 |
| Phenylephrine | 10.00 | 0.07 |
| Resin | 100.00 | 0.67 |
| Cyclodextrin | 45.00 | 0.30 |
| Glycerin | 20.00 | 0.13 |
| Hydrocolloid | XX | XX |
| Citric Acid | 30.00 | 0.20 |
| Xanthan Gum | 30.00 | 0.20 |
| Sucralose | 70.00 | 0.47 |
| Flavor | 600.00 | 4.00 |
| Edetate Disodium | 2.50 | 0.02 |
| Sodium Benzoate | 30.00 | 0.20 |
| Color | 10.00 | 0.07 |
| Purified water | 14042.50 | 93.62 |
| 25 mg/10 mg/20 mg15000 /dose Total | 15000.00 | 100 |

TABLE 11
Daytime Formula: Dextromethorphan HBr 20 mg/ Pectin 20 mg
liquid formulation with sodium benzoate, 0.5% at pH 4.0

| Ingredients | % w/v |
|---|---|
| Dextromethorphan hydrobromide | 0.133% |
| Beta cyclodextrin (Kleptose-beta-cyclodextrine) | 0.333% |
| Glycerin | 2.000% |
| Pectin 410 LA | 0.1330% |
| Sodium citrate dihydrate | 1.205% |
| Anhydrous citric acid | 1.1330% |
| Xanthan gum | 0.4000% |
| Sucralose NF | 0.4000% |

Flavor from Mixed berry family being optimized

| | |
|---|---|
| Edetate disodium | 0.0300% |
| Sodium benzoate | 0.5000% |
| Carmine PG 03410 | 0.0300% |
| Purified water | q.s. to 100% |

TABLE 12
Nighttime Formula: Diphenhydramine HCl 25 mg/ Pectin 20 mg
liquid formulation with sodium benzoate, 0.6% at pH 5.0

| Ingredients | % w/v |
|---|---|
| Diphenhydramine Hydrochloride | 0.167% |
| Beta cyclodextrin (Kleptose-beta-cyclodextrine) | 0.333% |
| Glycerin | 2.000% |
| Pectin 410 LA | 0.1330% |
| Sodium citrate dihydrate | 1.911% |
| Anhydrous citric acid | 0.6720% |
| Xanthan gum | 0.4000% |
| Sucralose NF | 0.4000% |

Flavor from Mixed berry family being optimized

| | |
|---|---|
| Edetate disodium | 0.0300% |
| Sodium benzoate | 0.6000% |
| Carmine PG 03410 | 0.0300% |
| Purified water | q.s. to 100% |

Below is an example of a taste-masking trial. Table 15 shows data from 15 subjects on various aspects of the formulas in Table 13 and 14. The formula in Table 13 is a daytime formula comprising both a cyclodextrin and a hydrocolloid for taste-masking, and the final pH was adjusted to 4.0. The formula in Table 14 is also a daytime formula, but does not include any taste-masking agents and the pH of the final formulation was not adjusted. The formula in Table 14 is the “control” as shown in Table 15. Fifteen subjects were chosen randomly to test both (blinded) formulations.

TABLE 13
Dextromethorphan HBr 20 mg/ Pectin 20 mg liquid
formulation with sodium benzoate, 0.5% at pH 4.0

| Ingredients | % w/v |
|---|---|
| Dextromethorphan hydrobromide | 0.133% |
| Beta cyclodextrin (Kleptose-beta-cyclodextrine) | 0.333% |
| Glycerin | 2.000% |
| Pectin 410 LA | 0.1330% |
| Sodium citrate dihydrate | 1.205% |
| Anhydrous citric acid | 1.1330% |
| Xanthan gum | 0.4000% |
| Sucralose NF | 0.4000% |

Flavor from Mixed berry family being optimized

| | |
|---|---|
| Edetate disodium | 0.0300% |
| Sodium benzoate | 0.5000% |
| Carmine PG 03410 | 0.0300% |
| Purified water | q.s. to 100% |

TABLE 14
Dextromethorphan HBr 20 mg/ Pectin 20 mg liquid formulation
with sodium benzoate, 0.5% at pH 6.0 to 7.0 (Control Formula)

| Ingredients | % w/v |
|---|---|
| Dextromethorphan hydrobromide | 0.133 |
| Glycerin | 2.000 |
| Xanthan gum | 0.4000 |
| Sucralose NF | 0.4000 |

Flavor from Mixed berry family being optimized

| | |
|---|---|
| Edetate disodium | 0.0300 |
| Sodium benzoate | 0.5000 |
| Carmine PG 03410 | 0.0300 |
| Purified water | q.s. to 100 |

TABLE 15

| | Control | | Daytime | |
|---|---|---|---|---|
| | Avg. Sample | | Avg. Sample | |
| Attributes/Ref# | Score | Comments | Score | Comments |
| Overall Liking (1-5) | 3.0 | Acceptable | 4.5 | Great viscosity |
| 1 = Dislike extremely | | viscosity | | |
| 5 = Like extremely | | | | |
| Bitter after taste Intensity | 1.5 | Very Bitter | 4.5 | Not bitter or |
| (1-5) | | | | very slightly |
| 1 = Very bitter | | | | bitter |
| 5 = Not at all bitter | | | | |
| Flavor liking (1-5) | 3.3 | Taste + smell | 4.2 | Nice berry smell |
| 1 = Dislike extremely | | | | |
| 5 = Like extremely | | | | |
| Flavor intensity (1-5) | 3.2 | Acceptable | 3.6 | Acceptable |
| 1 = Too weak | | | | |
| 3 = Just about right | | | | |
| 5 = Too strong | | | | |
| Sweetness Intensity (1-5) | 3.4 | Very sweet | 3.2 | Very sweet |
| 1 = Not at all sweet | | | | |
| 3 = Just about right | | | | |
| 5 = Too sweet | | | | |
| Sourness Intensity (1-5) | 1 | Not sour | 3.5 | Nice sourness |
| 1 = Not at all sour | | | | level |
| 5 = Very sour | | | | |

| | | |
|---|---|---|
| Preference Ranking from | Avg. preference ranking: 2.56 | Avg. preference ranking: 3.91 |
| most (5) to least (1) | Bitterness level: 1.5 (very bitter) | Bitterness level: 4.5 (not bitter) |
| Additional Comments | Room to work on bitterness | Berry flavor comes out. |
| | aspect | Bitterness is very less or not |
| | Bitter or unpleasant after taste | perceptible |
| | | No aftertaste |

Various formulations were prepared as shown in Table 16 below with reduced levels of Dextromethorphan HBr and pectin. The taste profiles of the formulations were evaluated by test subjects. The control Sample A was reported to have a bitter taste. Sample B did not taste bitter but was not sweet. Sample C did not taste bitter and was sweet. Sample D had a bitter aftertaste, believed to result from an excess of sucralose. Overall, Sample C was determined to be acceptable by most of the test subjects.

TABLE 16

| | Sample A | | | |
|---|---|---|---|---|
| | (Control) | Sample B | Sample C | Sample D |
| Ingredients | % w/v | % w/v | % w/v | % w/v |
| Dextromethorphan | 0.098 | 0.098 | 0.098 | 0.098 |
| hydrobromide | | | | |
| Beta cyclodextrin | 0.000 | 0.245 | 0.245 | 0.333 |
| (Kleptose-beta- | | | | |
| cyclodextrine) | | | | |
| Glycerin | 1.961 | 1.961 | 1.961 | 1.961 |
| Pectin 410 LA | 0.0980 | 0.0980 | 0.0980 | 0.0980 |
| Sodium citrate dihydrate | 1.205 | 1.205 | 1.205 | 1.205 |
| (Trisodium citrate) | | | | |
| Anhydrous citric acid | 1.133 | 1.133 | 1.133 | 1.133 |
| Xanthan gum | 0.400 | 0.400 | 0.400 | 0.400 |
| Sucralose NF | 0.000 | 0.000 | 0.294 | 0.400 |
| Nat FL Dark berry type | 1.000 | 1.000 | 1.000 | 1.000 |
| flavour# 540839A | | | | |
| Edetate disodium | 0.030 | 0.030 | 0.030 | 0.030 |
| Sodium benzoate | 0.500 | 0.500 | 0.500 | 0.500 |
| Carmine PG 03410 | 0.030 | 0.030 | 0.030 | 0.030 |
| Purified water | q.s. 100 | q.s. 100 | q.s. 100 | q.s. 100 |

### Methods of Manufacturing Pharmaceutical Compositions Comprising a Taste-masking Multi-Component Physical Barrier System

The multi-component taste-masking physical barrier described herein may be applied to one or more APIs using any of various coating techniques. For example, suitable techniques can include granulation, a liquid process, film coating, spray drying, and/or direct mixing.

Liquid Dosage Form Preparation:

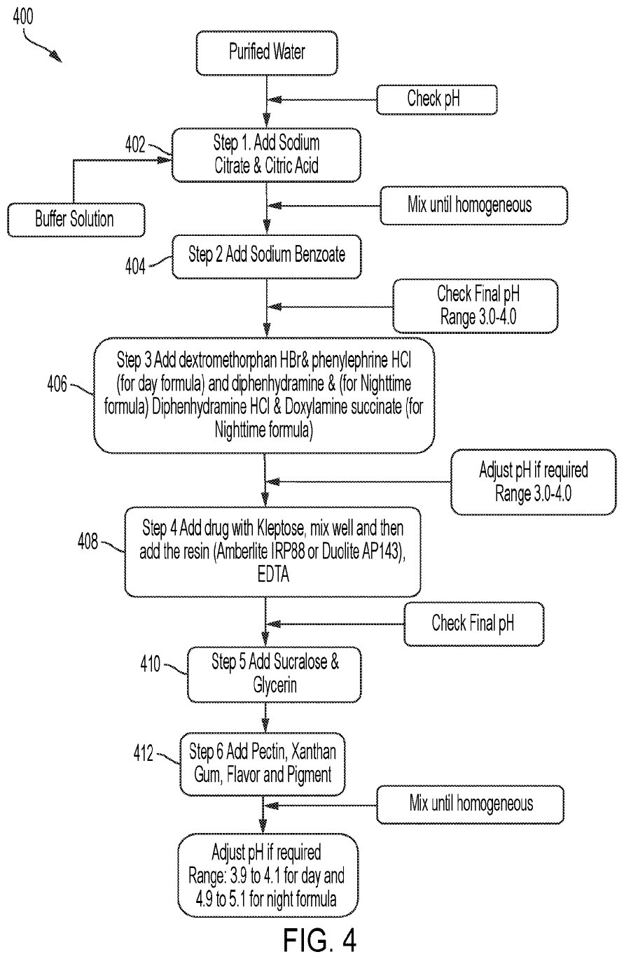

FIG. 4 shows an example of a process 400 for preparing a liquid pharmaceutical formulation comprising a multi-component taste-masking physical barrier, according to some embodiments. For example, the process 400 of FIG. 4 may be used to prepare the compositions provided in Tables 5 and/or 8.

Prior to step 402, the pH of purified water may be identified. Based on this result, at step 402, a buffer solution of sodium citrate and/or citric acid may be added to the purified water to control and stabilize the pH of the purified water. The buffering solution should be mixed with the purified water until a homogenous mixture is achieved.

At step 404, sodium benzoate may be added to the pH-controlled purified water. The pH of the mixture after the addition of the sodium benzoate may be checked. In some embodiments, the final pH of the mixture should be between 3 and 4.

At step 406, one or more APIs may be added to the mixture. For example, for a daytime liquid formulation, dextromethorphan HBr and phenylephrine HCl may be added. For a nighttime liquid formulation, diphenhydramine, diphenhydramine HCl, and/or doxylamine succinate may be added. If required, the pH may be adjusted after the addition of the one or more APIs.

At step 408, a hydrocolloid (e.g., Kleptose) may be added to the mixture and mixed well. Then, the resin (e.g., Amberlite IRP88, Duolite AP143) may be added along with EDTA. This mixture can then be mixed well, and the pH can again be checked.

At step 410, sucralose and glycerin may be added to the mixture and mixed well.

Finally, at step 412, pectin, xanthan gum, flavor, and pigment may be added to the mixture and mixed until a homogenous mixture is formed. The final pH may be adjusted if necessary. For example, a daytime formulation may have a target pH of 3.9-4.1, and a nighttime formulation may have a target pH of 4.9-5.1.

Solid Dosage Form Preparation:

In some embodiments, a solid dosage form may be prepared by combining one or more APIs (or a pharmaceutically-acceptable salt thereof), a cyclodextrin, an ion exchange resin, and water, forming a first mixture. This first mixture is homogenized, and a pH modifying agent is added to adjust the pH of the mixture. A hydrocolloid and one or more excipients are added to the first mixture. In some embodiments, the resulting solid dosage form is a powder or granules. In some embodiments, the one or more excipients can include one or more of a viscosity modifying agent or a suspending agent.

In some embodiments, the method for forming a solid dosage form can include forming a tableting mixture by adding an additional one or more excipients to the powder or granules. The tableting mixture can then be compressed, forming a tablet dosage form. The tablet dosage form can include a swallowable tablet, a chewable tablet, a sublingual tablet, a buccal tablet, an orally disintegrating tablet, or an effervescent tablet.

Solid/Semi-Solid Dosage Form Preparation:

In some embodiments, a solid or semi-solid dosage form may be prepared by one or more APIs, an ion exchange resin, a cyclodextrin, and water, forming a mixture. The mixture can be homogenized, and a pH modifying agent may be added to adjust the pH of the mixture. A hydrocolloid and one or more excipients may be added to the mixture. The mixture may then be heated and molded into a solid or semi-solid dosage form.

In some embodiments, the one or more excipients can include one or more of a viscosity modifying agent or a suspending agent. In some embodiments, the solid or semi-solid dosage form may be a lozenge, a soft chew tablet, an orally disintegrating film strip, or a chewable gel. In some embodiments, the cyclodextrin may be combined with the one or more APIs and water prior to the addition to the resin. In some embodiments, the cyclodextrin may be combined with the one or more APIs and water at the same time as the resin.

### Example: Granulation Process

1. Add desired amount of purified water into container 1.
2. Slowly add API, cyclodextrin and/or resin
3. Adjust pH is desired.
4. Add homogenizer or high shear mixing for a maximum of 1 hr.
5. Slowly add remaining excipients into container 2.
6. Add material from container 1 into container 2.
7. Granulate until homogeneous and desired particle size is achieved (not more than 500 microns)
8. Dry material to desired loss on drying (less than 2% moisture)
9. Add additional excipients as required and punch it into tablets.

### Example: Liquid Process

1. In container 1 place purified water and add API, Resin and/or Cyclodextrin.
2. Using homogenizer mix until homogeneous.
3. Adjust pH if desired.
4. Place the following materials into container 2 (antimicrobial agents, antioxidant,
buffering agent, chelating agent, sweetener(s) etc.
5. Add contents from container 1 into container 2 while mixing.
6. Slowly add hydrocolloid, viscosity agents, flavor and/or color.

### Example: Semi-Solid Process

1. In container 1 place purified water and add API, Resin and/or Cyclodextrin.
2. Using homogenizer mix until homogeneous.
3. Adjust pH if desired.
4. Place into container 1 materials (antimicrobial agents, antioxidant, buffering agent, chelating agent, sweetener(s) etc.
5. Slowly add hydrocolloid, viscosity agent, suspending agent, flavor and color.
6. Pour contents into molds to allow to cool.

### Methods for Treating

In some embodiments, provided herein are methods for treating one or more health conditions comprising administering a pharmaceutical composition described herein. In some embodiments, methods are provided for treating a cough and/or a cold, the method comprising administering to a patient an oral pharmaceutical composition comprising one or more of dextromethorphan or a pharmaceutically-acceptable salt thereof, phenylephrine or a pharmaceutically-acceptable salt thereof, or diphenhydramine or a pharmaceutically-acceptable salt thereof. In some embodiments, methods are provided for treating pain and/or a fever, the method comprising administering to a patient an oral pharmaceutical composition comprising one or more of naproxen or a pharmaceutically-acceptable salt thereof, phenylephrine or a pharmaceutically-acceptable salt thereof, or diphenhydramine or a pharmaceutically-acceptable salt thereof. In some embodiments, methods are provided for treating hay fever and/or upper respiratory allergies, the method comprising administering to a patient an oral pharmaceutical composition comprising diphenhydramine or a pharmaceutically-acceptable salt thereof.

The foregoing description sets forth exemplary systems, methods, techniques, parameters, and the like. It should be recognized, however, that such description is not intended as a limitation on the scope of the present disclosure but is instead provided as a description of exemplary embodiments.

Although the description herein uses terms first, second, etc. to describe various elements, these elements should not be limited by the terms. These terms are only used to distinguish one element from another.

1. An oral pharmaceutical composition comprising:
an active pharmaceutical ingredient or a pharmaceutically acceptable salt thereof;
a pH modifying agent;
an ion exchange resin;
a cyclodextrin;
a hydrocolloid; and
one or more pharmaceutically acceptable excipients.

2. (canceled)

3. The oral pharmaceutical composition of claim 1, wherein the pH modifying agent comprises a buffering agent.

4. The oral pharmaceutical composition of claim 3, wherein the pH modifying agent comprises one or more of lactic acid, acetic acid, citric acid, tartaric acid, phosphoric acid, or carbonic acid, or any pharmaceutically acceptable salt thereof.

5. The oral pharmaceutical composition of claim 1, wherein:
if the pharmaceutical composition is a liquid dosage form, the pharmaceutical composition has a pH from about pH 4 to about pH 7; or
if the pharmaceutical composition is a solid or semi-solid dosage form, the pharmaceutical composition has a pH from about pH 4 to about pH 7.

6. The oral pharmaceutical composition of claim 1, wherein the ion exchange resin is a cationic exchange resin.

7. The oral pharmaceutical composition of claim 1, wherein the ion exchange resin is an anionic exchange resin.

8. The oral pharmaceutical composition of claim 1, wherein the cyclodextrin comprises one or more of alpha-cyclodextrin, beta-cyclodextrin, or gamma-cyclodextrin.

9. The oral pharmaceutical composition of claim 1, wherein the cyclodextrin is beta-cyclodextrin.

10. The oral pharmaceutical composition of claim 1, wherein the cyclodextrin is hydroxypropyl beta-cyclodextrin.

11. The oral pharmaceutical composition of claim 1, wherein the hydrocolloid is pectin, carrageenan, xanthan gum, gellan gum, or any combination thereof.

12. The oral pharmaceutical composition of claim 11, wherein the hydrocolloid is pectin, xanthan gum, guar gum, or a combination thereof.

13. The oral pharmaceutical composition of claim 1, wherein the molar ratio of ion exchange resin to active pharmaceutical ingredient is at least about 0.5:1.

14. The oral pharmaceutical composition of claim 1, wherein the (molar/weight) ratio of cyclodextrin to active pharmaceutical ingredient is at least about 4:1.

15. The oral pharmaceutical composition of claim 1, wherein the (molar/weight) ratio of hydrocolloid to active pharmaceutical ingredient is at least about 0.5:1.0.

16. The pharmaceutical composition of claim 1, wherein the active pharmaceutical ingredient or a pharmaceutically acceptable salt thereof is present in both an ionized form and in a non-ionized form, and wherein the amount of the ionized form of the active pharmaceutical ingredient or a pharmaceutically acceptable salt thereof present in the pharmaceutical composition is less than the amount of the non-ionized form.

17. The pharmaceutical composition of claim 16, wherein the molar ratio of non-ionized active pharmaceutical ingredient to ionized active pharmaceutical ingredient is at least about 7,000 to 1 in synthetic saliva solution.

18. The pharmaceutical composition of claim 17, wherein the pharmaceutical composition comprises less of the ionized form of the active pharmaceutical ingredient or a pharmaceutically acceptable salt thereof as compared to a reference pharmaceutical composition, and
wherein the reference pharmaceutical composition does not contain at least two of a pH modifying agent, ion exchange resin, cyclodextrin and hydrocolloid.

19. (canceled)

20. The pharmaceutical composition of claim 1, wherein the pharmaceutical composition has an immediate release profile.

21. The pharmaceutical composition of claim 1, wherein the pharmaceutical composition has a delayed, sustained, or enteric release profile.

22. The pharmaceutical composition of claim 1, wherein the pharmaceutical composition is a liquid dosage form.

23-54. (canceled)
